(19) United States
(12) Patent Application Publication
(10) Pub. No.: US 2015/0051514 A1
(43) Pub. Date: Feb. 19, 2015

(54) CONCUSSION/BALANCE EVALUATION SYSTEM AND METHOD

(71) Applicant: Safety in Motion, Inc., Novi, MI (US)

(72) Inventors: David C. Stein, Troy, MI (US); Thomas F. Wallace, Fenton, MI (US); Priya Prasad, Plymouth, MI (US); Walter J. Borda, Northville, MI (US)

(21) Appl. No.: 14/460,457

(22) Filed: Aug. 15, 2014

Related U.S. Application Data
(60) Provisional application No. 61/866,063, filed on Aug. 15, 2013.
(60) Provisional application No. 61/866,071, filed on Aug. 15, 2013.

(51) Int. Cl. A61B 5/103; A61B 5/00; A61B 5/11

(52) U.S. Cl. CPC A61B 5/1036; A61B 5/1121; A61B 5/4076
USPC 600/595

(57) ABSTRACT

A balance testing apparatus includes a rigid bottom plate and a rigid top plate. At least three uni-axial load cells are coupled to the bottom plate and the top plate, and are configured to measure a set of predetermined parameters indicative of balance of an individual standing on the rigid top plate.

### CROSS-REFERENCE TO RELATED APPLICATIONS

This application claims the benefit of U.S. provisional patent application Ser. No. 61/866,063 filed Aug. 15, 2013, and further claims the benefit of U.S. provisional patent application Ser. No. 61/866,071 filed Aug. 15, 2013.

### FIELD OF THE INVENTION

The present technology relates to an apparatus and method for objectively clinically evaluating a subject's balance, and in particular, to an apparatus and method for evaluating a subject's balance within the field of diagnosing and evaluating traumatic brain injury and concussions.

### BACKGROUND OF THE INVENTION

A concussion or a concussive injury is a traumatic brain injury that alters the function of the brain and the central nervous system. Concussion effects are usually temporary, but can include problems with headache, concentration, memory, judgment, balance and coordination.

It is known that concussive injuries are caused by positive and negative acceleration forces experienced by the brain and may result from linear or rotational accelerations (or both). Both linear and rotational accelerations are likely to be encountered by the head at impact, damaging neural and vascular elements of the brain. These brain injuries can cause a loss of consciousness, but most concussions do not. As a result, it is possible to have experienced a concussive injury and not realize it.

Activities that pose a risk of head injury or that otherwise pose a risk of physical harm to the central nervous system and to the brain are subject to increasing scrutiny. Additionally, long-term effects of head and brain injuries are being extensively studied. Concussions may be experienced during activities where collisions between participants frequently occur (e.g., military activities, sporting events such as football, lacrosse, ice hockey, soccer and the like). In high-impact activities where deliberate collisions between participants occur, the potential for physical harm and/or injury is greatly enhanced. Although most concussions occur in high-impact activities, low-impact activities are not immune to mild traumatic brain injury, for which the short-term and long-term cumulative effects are not well understood. However, it is generally understood that a second concussive injury before recovery from a prior concussive injury can result in more significant injury and in prolonged dysfunction, especially in youth.

Thus, especially at the school level and as a result of legislation in many states, authorities have become sensitive to the risk of injury to which student participants are exposed, and to potential legal liability of the school system when injury results. Greater emphasis is therefore being placed on proper training and instruction to limit potential concussive injuries, and to applying objective criteria to any return-to-play (“RTP”) decisions.

However, objective criteria are often unavailable or are subject to manipulation. RTP decisions can be strongly influenced by supervisors, parents and coaches who want an individual to return to an activity. Because much of the assessment is subjective, outside influences, including apparent subjective acceptable performance by the patient in a clinical assessment may become especially persuasive. Moreover, even subjective diagnosis of concussive injury depends on accurate reporting by the potentially injured individual, who may out of enthusiasm or otherwise seek to return to the activity in spite of potential injury. Lastly, accurate diagnosis and RTP decision-making further depends on accurate and repeatable administration of accepted testing protocols. In some instances, appropriate medical staff and facilities may not be available due to cost or due to the fact that the potential injury may have occurred in a location remote from access to such medical staff and facilities, especially in non-professional sporting activities. RTP decisions have also come under scrutiny in professional sports, where even greater incentives exist for a valuable athlete to return to activity in spite of the possibility of having a concussive injury.

Surprisingly, contemporary health care and sports medicine lack sophisticated tools to easily, quickly and objectively assess brain function of patients suspected of experiencing concussive injury, especially in the immediate aftermath of such injury, and also over the longer recovery term, Physicians, trainers, and on-scene medical staff, if properly trained, currently utilize standard neurological examinations and cognitive questioning to subjectively determine the relative severity of the injury and its effect on the individual. If proper facilities and medical staff are present proximate the site of an injury, a patient's immediate mental and neurological status is typically assessed clinically by an interview and by a subjective physical exam, such as with a balance test.

Balance evaluation is known to be a leading indicator to diagnose possible concussive injury, and to evaluate the severity of such concussive injury. Balance testing is also increasingly utilized to manage sports-related concussive injuries. At present, there are several subjective test protocols that may be applied immediately post-injury to assess the balance of a potentially injured subject. Balance test protocols are described in the literature, and include the Balance Error Scoring System (“BESS”) test and modified BESS test, which differs from the BESS test in that testing is performed on a hard surface instead of on foam. Other balance-based tests include the SCAT3 test, and the NFL Sideline Concussion Assessment Tool, both of which utilize the modified BESS test. A further test called the Sensory Organization Test (“SOT”) applies balance tests similar to the BESS and the modified BESS test, but applies the balance tests only on a complex, expensive, and highly technical force plate system. The SOT test is considered by some to be the “gold standard” of balance testing, and has been used to show balance deficits that last several days post injury.

Balance test protocols are advantageous because they may be quickly and easily implemented, and can include low-cost testing that may be performed virtually anywhere within a short period of time. With respect to the BESS and modified BESS based protocols, however, interpretation of the results of the balance test protocols requires a subjective assessment of the patient's performance by the test administrator. The subjective assessment may lead to high variability between test applications and results for multiple tests applied to the same individual, even when successively applied within a short period of time, which may increase the difficulty of making accurate assessments of potentially injured individuals. Moreover, the subjective assessment may lead to high variability between results of tests administered or monitored by different individuals. For example, two individuals monitoring the same test being administered to a potentially injured individual may reach very different conclusions when observing the test. Thus, there is unacceptable subjective variability between tests administered to an injured person, and there is further unacceptable subjective variability between the same tests administered by different individuals to the same injured person.

The SOT test attempts to remove some of the subjective variability, but requires use of a known, non-movable platform. The SOT test and equipment is high precision, relatively expensive, and fixed in place, and is therefore not available for use as a portable tool. Thus, the SOT test is not generally available for immediate post-injury diagnosis and evaluation.

Accordingly, the provision of inexpensive and readily obtainable objective and sensitive methods to detect subtle brain dysfunction resulting from concussion is desirable.

Additionally, because of the variation in testing methodology and in testing equipment, test results obtained using a first device may not be valid or translate to equivalent test results obtained using a second device. Moreover, test results obtained in one location or at one time using a first type of device may not be valid or translate to results obtained in a second location at a second, later time, using the same or different types of devices. Apparatus and methods are desired to effectively compare test results obtained using devices that assess a test subject's balance to detect subtle brain dysfunction resulting from concussion is desirable

### SUMMARY OF THE INVENTION

Concordant and consistent with the present invention, an inexpensive and portable system and method for assessing and diagnosing concussive injury has surprisingly been discovered.

A balance testing apparatus includes a rigid bottom plate and a rigid top plate. At least three uni-axial load cells are coupled to the bottom plate and the top plate, and are configured to measure a set of predetermined parameters indicative of balance of an individual standing on the rigid top plate. The apparatus may include a processor, including data storage, coupled to each of the at least three uni-axial load cells and adapted to collect the measured set of predetermined parameters from each of the at least three uni-axial load cells. The data storage additionally may include an instruction set to enable the processor to correlate the measured set of predetermined parameters to the balance of the individual. A display device coupled to the processor is configured to display the measured set of predetermined parameters in a desired format. The rigid top plate and rigid bottom plate may be a composite material having negligible flex over a width and a length of the apparatus. Each of the at least three uni-axial load cells may be an automotive grade load cell.

In one embodiment of the system, the balance testing apparatus is portable and weatherproof, further including an exterior housing and at least one seal for isolating the at least three uni-axial load cells from an exterior environment. In another embodiment, one or more of the processor, the display device, the load cells and the accelerometers are wirelessly coupled. An internal battery or an external power supply may be provided to enhance the portability and operability of the force plate. In another embodiment, accelerometers may be mounted on the bottom plate to account for movement of the bottom plate relative to the top plate in both the X and the Y directions, to thereby maximize and validate an accuracy of the measured predetermined parameters. In one embodiment, the force plate includes an adjustment mechanism to balance the loading through the plurality of load cells and to establish a common plane for load cell measurement.

In one embodiment, the predetermined parameters include one or more of anterior-posterior (“A/P”) sway, lateral sway, center of gravity, center of pressure, weight, or the like, and may be derived using a predetermined regimen of simple balance tests. In another embodiment, the processor is configured to administer one of the NFL test, the SCATS test, the BESS test, and the modified BESS test.

A method for objectively measuring a subject's change in balance is also provided. The method includes measuring a baseline data set including predetermined parameters indicative of balance of an individual at a first time, measuring a second data set including predetermined parameters indicative of balance of the individual at a second time, and comparing the first and the second data sets to measure a change in the predetermined parameters between the baseline dataset and the second data set. The first time may occur before or immediately after a suspected injury, and the second time may be any time after the injury.

In one embodiment, the predetermined parameters include one or more of A/P sway, lateral sway, center of gravity, center of pressure, weight, or the like. In another embodiment, the method further includes the measuring a plurality of second data sets to track assess balance improvement and recovery of the individual. In another embodiment, the method further includes storing the first and second data sets of a plurality of individuals to create a database, and may further include transmitting the stored first and second data sets of the plurality of individuals to a long-term data storage for review and analysis. The long-term data storage may store data sets collected for an individual subject, a group of subjects, a class of subjects, or for all subjects.

A test fixture for assessing, comparing, validating and evaluating balance test devices is also provided. The test fixture includes at least one of a static test fixture and a dynamic test fixture. The static testing fixture is configured to apply a static force to at least a portion of a flat, level surface, such as a top surface of a force plate used for evaluating a test subject's balance. The static force may be applied over any area. In one embodiment, the static test fixture includes a fulcrum arm having a fixed first end and a free distal end. A fulcrum rest is spaced a predetermined distance from the fixed first end along the fulcrum arm. The fulcrum rest is configured to cover a predetermined area on a top surface of a force plate to apply a known force to a top surface of the force plate. The distal end of the fulcrum aim extends a preselected distance from the fulcrum rest. A processor is connected to the force plate to collect measurements of predetermined parameters indicative of a force exerted on the top plate by the static test fixture and to compare the measured predetermined parameters to the known force applied by the static test fixture.

The dynamic test fixture includes a base configured to rest on at least a portion of a top surface of a force plate. The base has a predetermined size and shape, and rests on the top surface of the force plate over a substantially planar surface having a known area. A reciprocating mass is coupled to the base plate. The reciprocating mass is constrained to limited degrees of freedom, and is configured to reciprocate along trajectories having known lengths and angles relative to the substantially planar surface to apply a known dynamic force to the top surface of the force plate. A processor is connected to the force plate to collect measurements of predetermined parameters indicative of a force exerted on the top plate by the dynamic test fixture and to compare the measured predetermined parameters to the known force applied by the static test fixture. In one embodiment, the reciprocating mass is mounted on a pendulum having a known arm length and angular displacement.

### BRIEF DESCRIPTION OF THE DRAWINGS

The above, as well as other advantages of the present invention will become readily apparent to those skilled in the art from the following detailed description, particularly when considered in the light of the drawings described herein.

FIG. 1 is a schematic elevational view of a portable and weatherproof force plate according to an embodiment of the invention.

FIG. 2 is a schematic cross-sectional view of a portable and weatherproof force plate according to an embodiment of the invention.

FIG. 3 is a schematic elevational view of a static test fixture, according to an embodiment of the invention.

FIG. 4 illustrates in graphical form an exemplary set of measurements of predetermined parameters indicative of a force exerted on the top plate collected by the static test fixture according to an embodiment of the invention.

FIG. 5 is a schematic elevational view of a dynamic test fixture, according to an embodiment of the invention.

FIG. 6 illustrates in graphical form an exemplary set of measurements of predetermined parameters indicative of a force exerted on the top plate collected by the dynamic test fixture according to an embodiment of the invention.

### DETAILED DESCRIPTION

The following detailed description and appended drawings describe and illustrate various embodiments of the invention. The description and drawings serve to enable one skilled in the art to make and use the invention, and are not intended to limit the scope of the invention in any manner. Regarding methods disclosed, the order of the steps presented is exemplary in nature, and thus, the order of the steps can be different in various embodiments where possible.

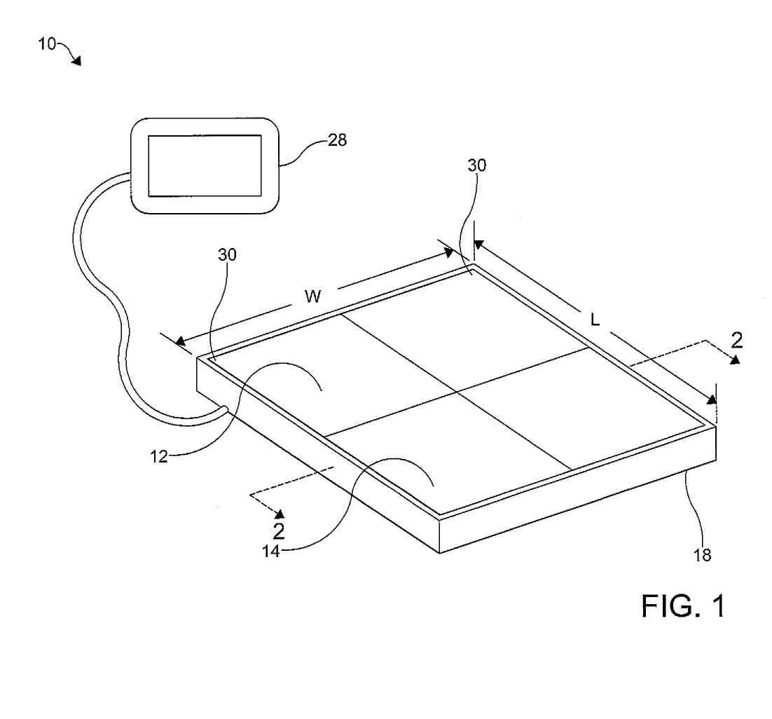

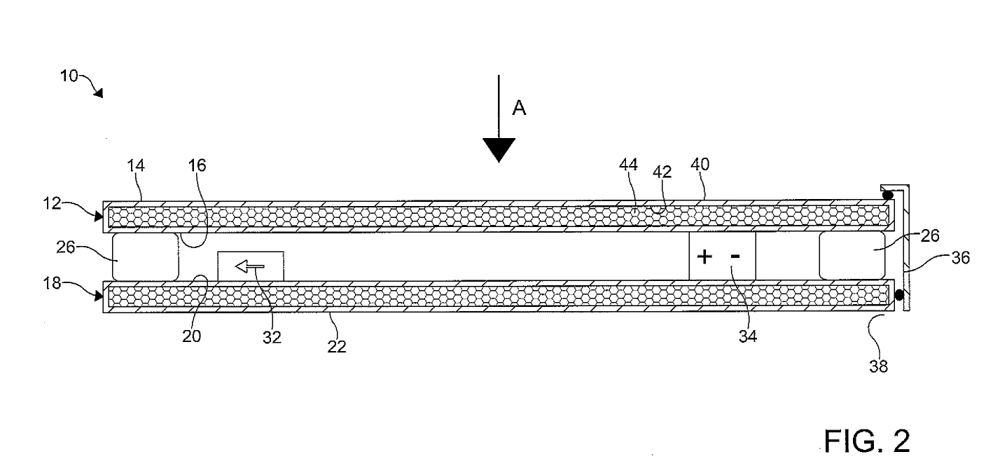

A force plate apparatus 10 according to the present invention is described with reference to FIGS. 1 and 2. The force plate 10 includes a rigid top plate 12 having a top surface 14 and a bottom surface 16. The force plate 10 additionally includes a rigid bottom plate 18 having a top surface 20 and a bottom surface 22. The top surface 14 of the top plate 12 and the top surface 20 of the bottom plate 18 define parallel planes. It is understood that the top plate 12 and the bottom plate 18 may have any shape and size, and may be made of any sufficiently lightweight, stiff and rigid material. “Rigid”, in this context, means that the top plate 12 and the bottom plate 18 are configured to minimize any substantial flex of the top plate 12 or the bottom plate 18 in the direction indicated by arrow A of FIG. 2 when a test subject stands on the top surface 14 of the top plate 12. Favorable results have been achieved when the top plate and the bottom plate are constructed of composite, laminate or other lightweight structural materials, such as engineered aluminum sheeting, where the top plate 12 and the bottom plate 18 flex less than 0.010 inches. Failure to minimize the flex of the top plate results in some of the force caused by the weight of the test subject to be taken up by the flex of the top plate, or may cause vibration or oscillation of the top plate, resulting in inaccurate readings at one or more of the load cells 26, and further resulting in inaccurate test results. Substantial flexing of the top plate 12 may also affect the balance of the test subject.

As best seen in FIG. 2, the top plate 12 and the bottom plate 18 may be formed of a composite having a top sheet 40 and a bottom sheet 42 formed of a first material, with an intermediate material 44 interposed between the top sheet 40 and the bottom sheet 42. As a non-limiting example, a top plate 12 and a bottom plate 18 may be formed from a panel having a top sheet 40 and a bottom sheet 42 constructed from milled aluminum sheet metal having a known thickness, each adhered, via epoxy, welding, or other known method, to an intermediate panel 44 formed from a honeycomb-shaped aluminum alloy having known thickness density and perforation spacing. As a further non-limiting example, the top plate 12 and the bottom plate 18 may be formed from a graphite composite, a glass composite, or other known composite to make a rigid top plate 12 and bottom plate 18.

The top plate and bottom plate are shown in FIGS. 1 and 2 as being generally rectangular with a length L and a width W, thereby defining a size of the force plate apparatus 10, and in particular, the size of the top plate 12. It is understood that the bottom plate 18 may have any size, including length L and width W, sufficient to support the force plate apparatus 10, but the best results are obtained where the top plate 12 and the bottom plate 18 are the same size. Additionally, the size of the force plate apparatus 10 may be adjusted to fit the foot and/or shoe size of potential subjects that may stand on the top plate 12 during balance testing. For example, the size of the force plate apparatus 10 may be smaller for children than for adults. However, favorable results have been obtained where the top plate and bottom plate measure 20 inches in width, and 30 inches in length.

Like the top plate 12, the bottom plate 18 is a flat plate and does not include feet. By using a flat bottom plate 18, the bottom plate 18 is used to establish a reference plane for the force plate. Establishing a reference plane is desirable because it allows for the force plate apparatus to take measurements on a wide variety of surfaces, such as on soft or uneven ground. Moreover, the reference plane allows for measurements to be taken when the force plate apparatus is inclined at an angle. As a non-limiting example, use of a flat bottom plate 18 enables achievement of favorable results when the force plate apparatus is inclined at up to a 15 degree angle from level. It is understood, however, that significant incline of the force plate apparatus may interfere with the ability of a test subject to perform balance testing.

Because three points are required to define a plane of the top plate 12, the force plate apparatus 10 shown in FIGS. 1 and 2 includes at least three uni-axial load cells 26. The at least three uni-axial load cells 26 are coupled to the bottom surface 16 of the top plate 12 and to the top surface 20 of the bottom plate 18. Because the force plate apparatus 10 is generally rectangular, favorable results have been achieved using four uniaxial load cells coupled to the top plate 12 and to the bottom plate 18 of the top plate 12. In one embodiment, the load cells are affixed to one of the top plate 12 and the bottom plate 18, and are “soft” mounted to the other of the top plate 12 and the bottom plate 18. As non-limiting examples, the “soft” mount may include a removable coupling, and may further include simply resting the top plate 12 on the uni-axial load cells 26 within a housing that constrains movement of the top plate 12 and the bottom plate 18. Use of “soft” mount of a load cell to a plate eliminates off-axis loading through the load cell. Additionally more than three uni-axial load cells may be used to increase the weight tolerance of the force plate or to improve the accuracy and resolution of the balance assessment, but are not required. Optionally, if more than three uni-axial load cells are utilized, one or more of the uni-axial load cells may include a height adjustment mechanism to enable substantially planar alignment between the load cells 26, thereby resulting in a flat, substantially planar top surface of the top plate

Each load cell 26 is mounted at a known position proximate each corner 30 of the force plate apparatus 10. When a test subject stands on the upper surface 14 of the force plate apparatus 10, each load cell 26 therefore bears a known proportion of the weight of the test subject, measured directly and continuously by each load cell 26. During any dynamic test, such as a balance test administered to an individual, the proportion of weight borne by each load cell 26 continuously changes as the balance of the test subject shifts. Each load cell 26 is further connected to a processor 28 adapted to collect the weight measurements of each load cell over a specified duty cycle. The processor 28 may be a dedicated computer device wired to the force plate apparatus 10, or may be a separate computer device, such as a laptop or desktop computer, coupled to the load cells 26 wirelessly or by wires.

From the collected data, the processor 28 is able to quickly, continuously and accurately calculate predetermined parameters indicative of balance of the test subject. As non-limiting examples, measured predetermined parameters may include one or more of anterior-posterior sway, lateral sway, center of gravity, center of pressure, weight, or the like, and may be derived using a predetermined regimen of simple balance tests. Additionally, the processor may be adapted to administer known and accepted balance tests, such as the NFL test, the SCAT3 test, the BESS test, and the modified BESS test. Faults or mistakes may be noted and recorded by the processor using the same criteria applied to each test subject, so that variability between test results is minimized, while accuracy of the tests is maximized. As a non-limiting example, tests and evaluations using the inventive force plate apparatus have shown a better than 90% correlation between errors observed by a panel of test administration experts and the processor noting and recording those errors. As a further non-limiting example, the balance assessment system may be used to administer the modified Balance Error Scoring System test protocol (the “mBESS protocol”). According to the mBESS protocol, a test subject is required to perform three separate balance tests, each balance test having a duration of twenty seconds. During application of each balance test, the balance assessment system of FIG. 2 is capable of objectively measuring the predetermined parameters at a desired frequency to acquire a very large data set within the twenty second test application. For example, a twenty second balance test may yield 50,000 data points when measured at 2.5 kHz. The number of data points collected is limited only by sampling frequency and processor speed.

Accelerometers 32 may be mounted on the bottom plate 18 to account for movement of the bottom plate 18 relative to the top plate 12 in both the X and Y planes, to thereby maximize and validate an accuracy of the measured predetermined parameters. Because the bottom plate 18 movement relative to top plate 12 movement is accounted for, the force plate apparatus 10 of the present invention is capable of being used on uneven or soft ground, and does not require a hard, level surface for accuracy. As long as the load cells 26 are accurately placed and properly calibrated, the force plate apparatus 10 may be constructed using inexpensive off-the-shelf components having known specifications. By using off-the-shelf components, a cost of the force plate apparatus 10 may be minimized. The load cells 26 used in the force plate may be capable of sensing subject of any weight, and may be adjusted to any desired weight range. As a non-limiting example, the force plate may be calibrated to sense subjects weighing between 40 and 400 pounds. Additionally, an internal power supply 34, such as a rechargeable battery, may optionally be provided to enhance the portability of the force plate apparatus 10.

All of the components of the force plate shown in FIGS. 1 and 2 may be encased in a lightweight, weatherproof and impact resistant manner. The weatherproof and impact resistant encasement may further be provided with a non-slip surface on the top surface 14 of the top plate 12 for stability of the test subject. Thus, as shown in FIG. 2, the force plate apparatus may include an exterior housing 36 and at least one seal 38 interposed between the exterior housing 36, the top plate 12 and the bottom plate 18. The exterior housing 36, in combination with the seal 38 create a weather-resistant outer shell while shielding the load cells and any other component mounted to either the top plate 12 or the bottom plate 18 from exterior conditions, including weather and humidity. In an embodiment, the seal 38 is interposed between an outer perimeter of the bottom plate 18 and the exterior housing 36, and is configured to allow the bottom plate 18 to move independently of the exterior housing 36. Alternatively, the seal 38 may be configured in any way to allow the bottom plate 18 to move independently of the top plate 12.

The force plate apparatus 10 of the present invention therefore provides an inexpensive, portable, weatherproof and easy to use objective measuring device for use in the field to augment known balance assessment protocols currently utilized for subjective concussive injury assessment. Because the force plate apparatus 10 may easily and quickly acquire large data sets during relatively short balance test intervals, a balance assessment system may be repeatedly used to obtain baseline data sets for any test subject that may potentially be exposed to concussive injury at any later time. Additionally, a test subject may be asked to provide regularly scheduled balance test baseline data sets useful for later evaluations for possible concussive injury. Upon suffering a possible concussive injury, the test subject may perform a balance test protocol to provide a second data set. The balance assessment system may then quickly, easily and inexpensively provide a comparison between the baseline data sets and the second data set to objectively provide an evaluation of any change in the predetermined parameters between the baseline data set and the second data set. The measured change may be useful to maximize an accuracy of diagnosing a possible concussive injury. If a concussive injury is diagnosed, one or more additional data sets may be measured over any time interval, such as during a recovery period. The one or more additional data sets may be easily, quickly and inexpensively compared to the baseline data sets and to the second data set to assess improvement and recovery from a concussive injury.

Additionally, the processor 28 may include a data storage component. As noted above, the force plate apparatus 10 of the present invention may be used to collect large amounts of objective data for a plurality of test subjects. Because most previous methods for using balance testing to diagnose concussive injury applied subjective interpretation of test results, an insignificant amount of objective test data is available for research and assessment of concussive injury. The force plate apparatus 10 of the present invention may include sufficient data storage to the collect objective balance data useful for diagnosing concussive injury for use in research and assessment of concussive injuries. Over time, as more data sets are accumulated in the data storage component, the amount of data collected will increase, and may be useful for advancing the state of the art related to balance testing assessment of concussive injury. According to one embodiment, the data collected may include an expanded set of objectively measured predetermined parameters of a plurality of test subjects. Additionally, because the force plate apparatus measures weight and movement of the weight of a test subject, the processor 28 may be adapted and instructed to derive expanded data from the test subject. As non-limiting examples, the processor 28 may be adapted to derive one or more of anterior-posterior sway, lateral sway, center of gravity, center of pressure, weight, height, age, gender, position, test subject identification information or codes, impact characterization, question and answer responses, or the like. Thus, the force plate apparatus 10 may be used to collect any number of objective data sets and additionally may be configured to transmit all collected data sets to a long-term data storage for review and analysis. The long-term data storages may store data sets collected for an individual subject, a group of subjects, a class of subjects, or for all subjects.

It is also possible to utilize multiple force plate apparatuses 10 in different locations to collect data sets for a plurality of test subjects. When using many different force plate apparatuses 10, it is necessary to ensure that a data set acquired on one force plate apparatus is repeatable and applicable to any of the other force plate apparatuses used to collect the data sets to prevent a mismatch of data. In other words, it is necessary to validate each force plate apparatus to ensure that each force plate apparatus is measuring within known tolerances so that each data set may be used in a valid comparison.

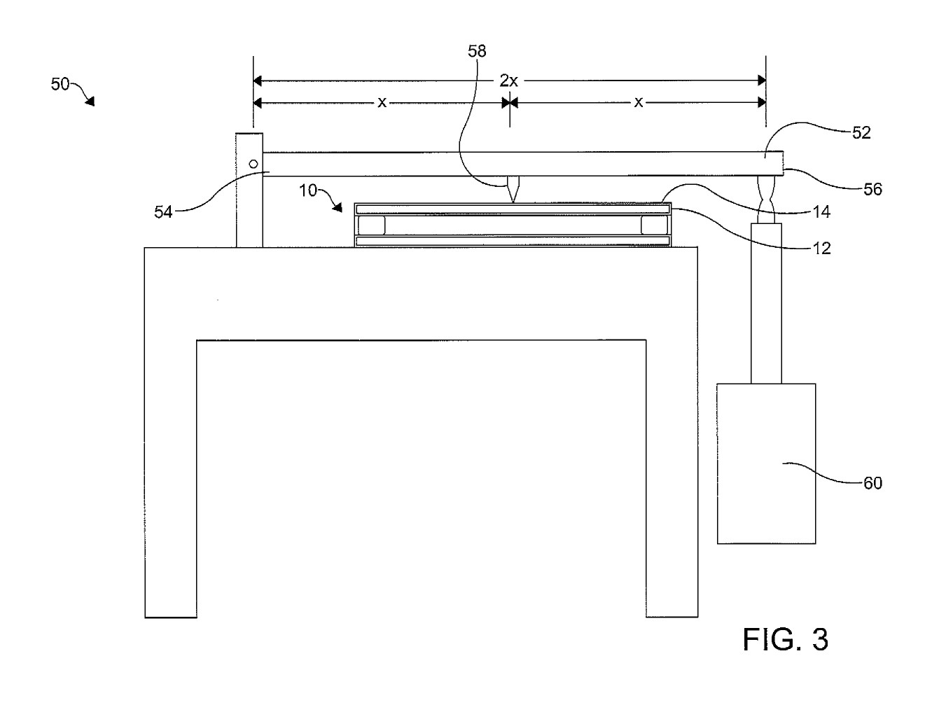

A static test fixture 50 useful for validating a force plate apparatus 10 is shown in FIG. 3. A force plate apparatus 10 is placed within a static test fixture 50, which is configured to apply a static force to at least a portion of a flat, level surface, such as a top surface 14 of a top plate 12 of the force plate apparatus 10. The static test fixture includes a fulcrum arm 52 having a fixed first end 54 and a free distal end 56. As shown, the fulcrum arm 52 lies in a plane substantially parallel to a plane defined by the top surface 14 of the force plate apparatus 10. It is understood, however, that the fulcrum arm 52 may lie at any known angle with respect to the plane defined by the top surface 14 of the force plate apparatus 12. A fulcrum rest 58 is spaced a first predetermined distance from the fixed first end 54 along the fulcrum arm 52. The fulcrum rest 58 is configured to cover a predetermined area on the top surface 14 of the force plate apparatus 10 to translate a force to the top surface 14 of the force plate apparatus 10. Favorable results have been obtained when the fulcrum rest 58 terminates in a small, known area, such as in a small ball bearing or a stylus point. As a non-limiting example, the fulcrum rest may terminate in a ⅜ inch ball hardened bearing, through which the force applied through the static test fixture 50 is translated to the force plate apparatus 10.

The distal end 56 of the fulcrum arm 52 extends a second preselected distance from the fulcrum rest 58, such that a force exerted against the distal end, such as, for example, by a weight 60 affixed to or suspended from the distal end 56, is amplified by a known amplification factor at the fulcrum rest 58 to cause a multiplied force to be exerted to the predetermined area on the top surface of the force plate apparatus 10. In FIG. 3, the fulcrum rest 58 is spaced a first distance x from the fixed first end 54. The distal end 56 of the fulcrum arm 52 is spaced a distance x from the fulcrum rest 58. The length of the fulcrum arm is therefore 2x. In this configuration, a weight 60 suspended from the distal end 56 of the fulcrum arm 52 results in double the weight 60 being applied to the top surface 14 of the force plate apparatus 10 through the fulcrum rest 58. It is understood that the fulcrum arm 52 may have any desired length and configuration, and that the configuration shown in FIG. 3 is merely exemplary.

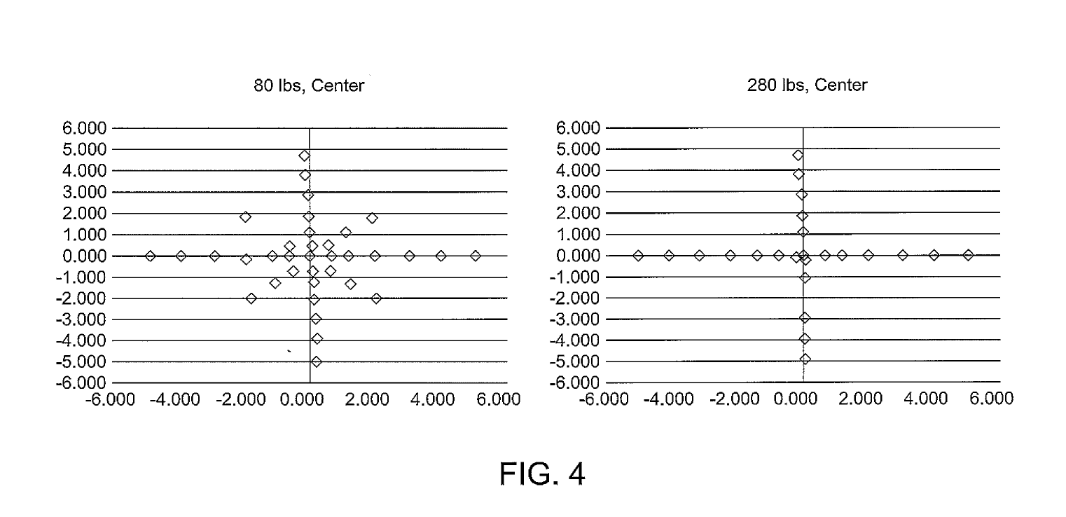

The processor 28 connected to the force plate apparatus 10 may be used to collect data sets while applying the static test fixture 50 to ensure performance of each force plate apparatus 10 within predetermined tolerances. Examples of such measurements are shown in FIG. 4. Additionally, FIG. 4 illustrates that measurements using the static test fixture 50 may be taken at a plurality of known points along the top surface 14 of the force plate apparatus 10. The measurements shown in FIG. 4 may also be compared against known actual locations at which known forces are exerted, to allow for evaluations of accuracy and tolerance of a given force plate. Additionally, similar measurements may be taken between more than one force plate, of similar or different configurations, to establish correlational characteristics between the more than one force plates, and to establish comparisons or validations of measurements made between the various force plates.

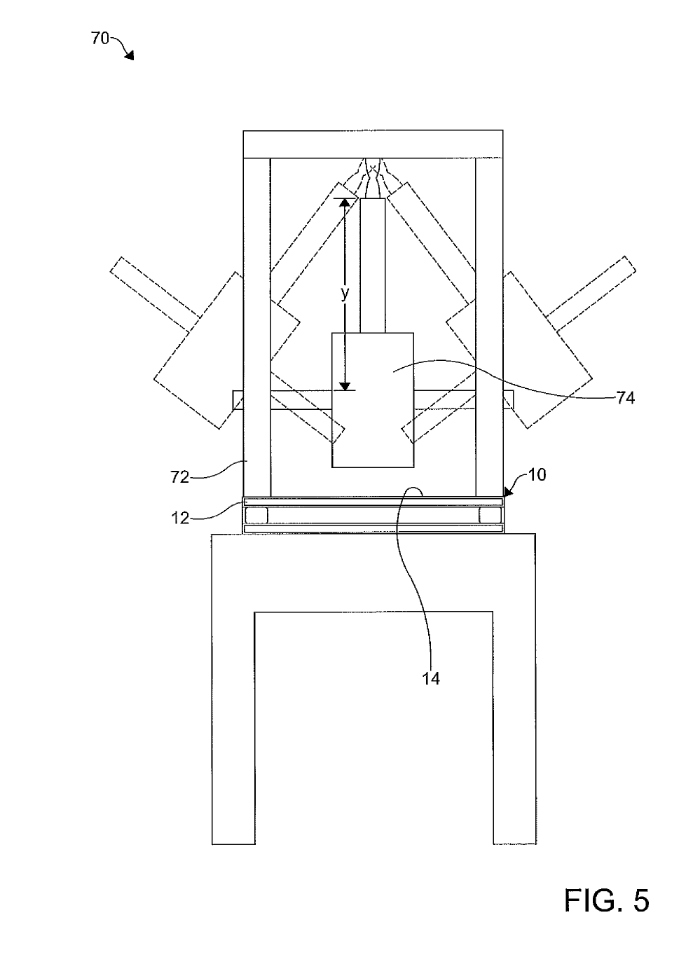

A dynamic test fixture 70 is shown in FIG. 5. The dynamic test fixture 70 includes a base 72 configured to rest on at least a portion of the top surface 14 of a force plate apparatus 10. The base 72 has a predetermined size and shape, and rests on the top surface 14 of the force plate apparatus 10 over a substantially planar surface having a known area. A reciprocating weight 74 is coupled to the base 72.

In FIG. 5, the reciprocating weight 74 is shown as a pendulum having a known mass, a known moment arm lengthy, and a known trajectory relative to the plane defined by the top surface 14 of the force plate apparatus 10. The dynamic test fixture 70 may further include instrumentation to precisely determine the location and trajectory of the reciprocating mass for comparison to measured results.

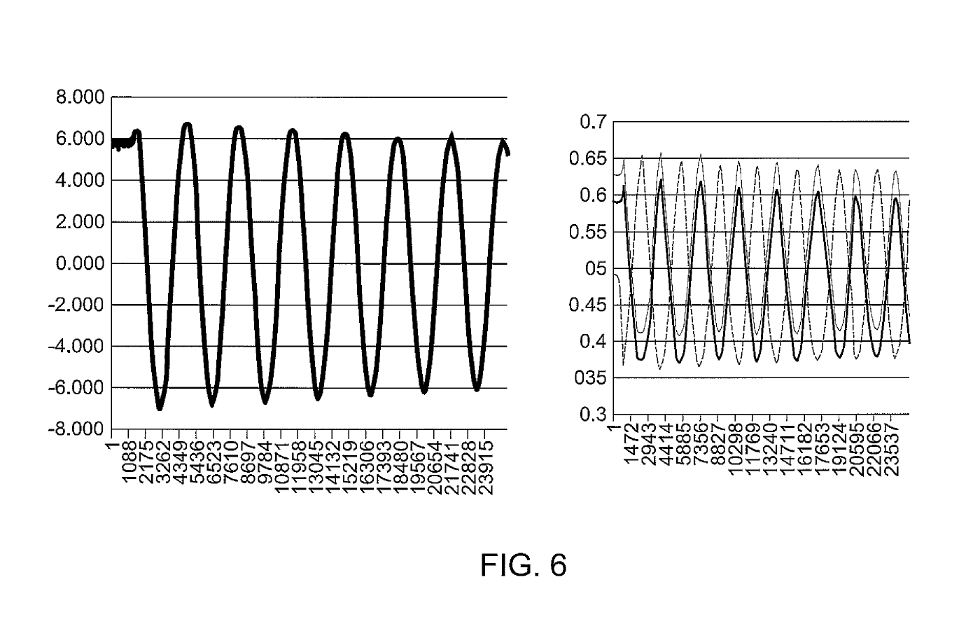

The processor 28 connected to the force plate apparatus 10 may be used to collect data sets while applying the dynamic test fixture 70 to ensure performance of each force plate apparatus 10 within predetermined tolerances. As a non-limiting example, an effective loading point of the Center of Gravity of the reciprocating weight 74 may be determined at any point through the prescribed arc of the reciprocating weight 74 because the length and angle of the moment arm from vertical is known, as is the mass of the reciprocating weight 74. FIG. 6 shown a comparison of measurements taken for the reciprocating mass position and the predetermined parameters measured by the force plate. The measurements shown in FIG. 6 may be compared against known actual locations at which known forces are exerted, to allow for evaluations of accuracy and tolerance of a given force plate. Alternatively, the static test fixture 50 and the dynamic test fixture 70 may be used to collect any number of objective data sets and may be configured to provide comparative analysis of a plurality of testing devices for consumer review or for competitive analysis. Additionally, similar measurements may be taken between more than one force plate, of similar or different configurations, to establish correlational characteristics between the more than one force plates, and to establish comparisons or validations of measurements made between the various force plates.

Accordingly, the static test fixture 50 and the dynamic test fixture 70 may be utilized separately or in combination to provide an objective evaluation, validation and comparison device for evaluating, validating, and comparing various testing devices used in the field to collect objective data useful for diagnosing concussive injury for use in research and assessment of concussive injuries. Appropriate correction factors may then be applied to each of the collected data sets to allow for direct comparison and/or normalization of each of the collected data sets based upon characteristics specific to each apparatus used to collect the data set. Alternatively, the static test fixture and the dynamic test fixture may be utilized to ensure that each force plate apparatus functions within predetermined tolerance to enable direct comparison of each collected data set without normalization thereof. When used in conjunction with the force plate apparatus of the present invention, the test fixtures provide a method for objectively measuring predetermined parameters of a plurality of testing devices and comparing the measurements against either actual conditions, or against similar or different measurement devices.

From the foregoing description, one ordinarily skilled in the art can easily ascertain the essential characteristics of this invention and, without departing from the spirit and scope thereof, make various changes and modifications to the invention to adapt it to various usages and conditions.

What is claimed is:

1. A balance testing apparatus, comprising:
a rigid bottom plate;
a rigid top plate;
at least three uni-axial load cells coupled to the bottom plate and the top plate, the at least three uni-axial load cells configured to measure a set of predetermined parameters indicative of balance of an individual standing on the rigid top plate.

2. The apparatus of claim 1, further comprising:
a processor, including data storage, coupled to each of the at least three uni-axial load cells adapted to collect the measured set of predetermined parameters indicative of balance of the individual;
wherein the data storage includes an instruction set to enable the processor to correlate the measured set of predetermined parameters to the balance of the individual.

3. The apparatus of claim 2, further comprising a display device coupled to the processor configured to display the measured set of predetermined parameters in a desired format.

4. The apparatus of claim 2, wherein the processor is configured to administer one of the NFL test, the SCAT3 test, the BESS test, and the modified BESS test.

5. The apparatus of claim 3, further comprising at least one accelerometer coupled to the bottom plate and configured to account for movement of the bottom plate relative to the top plate in both the X and the Y directions.

6. The apparatus of claim 5, wherein the processor, the display device, the load cells and the accelerometers are wirelessly coupled together.

7. The apparatus of claim 1, wherein the rigid top plate and the rigid bottom plate are formed from a composite material.

8. The apparatus of claim 1, wherein each of the uni-axial load cells is an automotive grade load cell.

9. The apparatus of claim 1, further comprising an exterior housing and at least one seal for isolating the at least three uni-axial load cells from an exterior environment.

10. The apparatus of claim 1, wherein the predetermined parameters include one or more of anterior-posterior sway, lateral sway, center of gravity, center of pressure, and weight.

11. A balance testing apparatus, comprising:
a rigid bottom plate;
a rigid top plate;
at least three uni-axial load cells coupled to the bottom plate and the top plate, the at least three uni-axial load cells configured to measure a set of predetermined parameters at a data measurement frequency indicative of balance of an individual standing on the rigid top plate;
a processor, including data storage, coupled to each of the at least three uni-axial load cells adapted to collect the measured set of predetermined parameters indicative of balance of the individual;
wherein the data storage includes an instruction set to enable the processor to correlate the measured set of predetermined parameters to the balance of the individual.

12. The apparatus of claim 11, wherein the processor is configured to administer one of the NFL test, the SCAT3 test, the BESS test, and the modified BESS test.

13. The apparatus of claim 11, further comprising at least one accelerometer coupled to the bottom plate and configured to account for movement of the bottom plate relative to the top plate in both the X and the Y directions.

14. The apparatus of claim 11, wherein the rigid top plate and the rigid bottom plate are formed from a composite material.

15. The apparatus of claim 11, further comprising an exterior housing and at least one seal for isolating the at least three uni-axial load cells from an exterior environment.

16. The apparatus of claim 11, wherein the predetermined parameters include one or more of anterior-posterior sway, lateral sway, center of gravity, center of pressure, and weight.

17. A method for balance testing, comprising:
measuring predetermined parameters indicative of balance of a test subject, wherein the measuring step is performed using a force plate, the force plate comprising:
a rigid bottom plate;
a rigid top plate; and
at least three uni-axial load cells coupled to the bottom plate and the top plate, the at least three uni-axial load cells configured to measure a set of predetermined parameters indicative of balance of an individual standing on the rigid top plate;
administering a known balance test protocol; and
evaluating the measured predetermined parameters according to the known balance test protocol to obtain a balance test protocol score.

18. The method of claim 17, wherein the known balance test protocol is one of the BESS test, the modified BESS test, the NFL test, and the SCAT3 test.

19. The method of claim 17, wherein the predetermined parameters are one or more of anterior-posterior sway, lateral sway, center of gravity, center of pressure, and weight.

20. The method of claim 17, wherein the balance test protocol score correlates to concussion in the test subject.
